(19) United States
(12) Patent Application Publication
(10) Pub. No.: US 2023/0260641 A1
(43) Pub. Date: Aug. 17, 2023

(54) METHODS OF HANDLING MEDICAL EQUIPMENT STATUS INFORMATION, AND SYSTEM

(71) Applicant: Olympus Winter & Ibe GmbH, Hamburg (DE)

(72) Inventors: Ralf Tessmann, Hamburg (DE); Sascha Jaskola, Hamburg (DE); Juri Sverdlov, Hamburg (DE); Ralf Siegmund, Ahrensburg (DE); Veronika Stefka, Hamburg (DE); Christian Karstens, Hamburg (DE)

(73) Assignee: Olympus Winter & Ibe GmbH, Hamburg (DE)

(21) Appl. No.: 18/109,965

(22) Filed: Feb. 15, 2023

Related U.S. Application Data
(60) Provisional application No. 63/310,173, filed on Feb. 15, 2022.

(51) Int. Cl. G16H 40/40

(52) U.S. Cl. CPC G16H 40/40

(57) ABSTRACT

A method of handling medical equipment status information. The method includes: receiving status information data from two or more pieces of medical equipment, wherein the status information data is received in a data format specific for each of the pieces of medical equipment; determining that the status information data indicate an error state of the pieces of medical equipment; assigning to each error state one or more global error categories of a predetermined list of global error categories, the global error categories not being specific to a certain piece of medical equipment among the pieces of medical equipment; and outputting error information data. The error information date including: identification data identifying a specific piece of medical equipment among the pieces of medical equipment, status information data received from the specific piece of medical equipment, and the one or more global error categories assigned to the detected error status.

### CROSS-REFERENCE TO RELATED APPLICATION

The present application is based upon and claims the benefit of priority from U.S. Provisional Application No. 63/310,173, filed on Feb. 15, 2022, the entire contents of which is incorporated herein by reference.

### BACKGROUND

### Field

The present disclosure relates to methods of handling medical equipment status information. More specifically, the present disclosure is related to handling of status information of medical or surgical endoscopes, and of endoscope reprocessing machines.

### Prior Art

Medical and surgical endoscopes are used in modern medicine for examination and treatment of patients suffering a widespread spectrum of medical conditions. Such endoscopes are highly complicated and expensive instruments. Therefore, other than many pieces of medical equipment, endoscopes are not discarded after single use, but are re-used many times after undergoing careful reprocessing procedures.

The reprocessing procedures are designed to remove dirt, stains, pathogens, and residues from an endoscope prior to re-use, so that cross-contamination of patients can safely be prevented. While some steps of the reprocessing procedures include manual operation, most of the steps are executed by automated endoscope reprocessing machines, like those provided by the applicant under the name “ETD”. Depending on the specific reprocessing procedures used, reprocessing may also include drying of endoscopes in drying cabinets. In the context of the present disclosure, the term “endoscope reprocessing machine” may include such drying cabinets.

To ensure that a surgical device like an endoscope is safe for re-use, reprocessing procedures are automatically monitored, and the results are stored in a database. Such results can include data specific to a single endoscope, like results of channel patency tests, endoscope leakage tests, or the like, and data specific to a reprocessing machine, like dosing volumes of detergents or disinfectants, processing temperature levels, duration of rinse cycles, or the like.

If any parameter is outside of a specified range, an error message is generated, indicating that an endoscope is not ready for use after reprocessing, and/or that a reprocessing machine requires servicing. Typically, such error messages include an alphanumeric error code specific to the type of reprocessing machine in which the error occurred, sometimes accompanied with a short plain text description.

In many hospitals and larger endoscopy offices, a plurality of reprocessing machines are installed, sometimes including reprocessing machines of different types. Status information from these machines is collected in a central control system, and is displayed to a user through a user interface.

For a user, it may be difficult to understand the nature and severity of an error message due to the different types of reprocessing machines, and the different error codes provided by different reprocessing machines.

It would be beneficial to improve handling of the status information of medical equipment so that a user can easier understand the nature of an error state and can initiate appropriate countermeasures.

It would further be beneficial to improve handling of status information of medical equipment so that upcoming issues of a piece of equipment can be identified before the piece of equipment goes into an error state.

It would also be beneficial to improve handling of status information of medical equipment so that it can more easily be determined whether an error resulted from a machine malfunction or from human error.

### SUMMARY

The present disclosure provides a method of handling medical equipment status information, the method comprising: receiving status information data from one or more pieces of medical equipment, wherein the status information data is received in a data format specific for each piece of medical equipment; determining that the status information data indicate an error state of the one or more pieces of medical equipment; assigning to each error state one or more global error categories of a predetermined list of global error categories, the global error categories not being specific to a certain piece of medical equipment; and outputting error information data comprising identification data identifying a specific piece of medical equipment, status information data received from the specific piece of medical equipment, and the one or more error categories assigned to the detected error status.

By outputting the one or more global error categories in case of a piece of medical equipment being in an error state, it is easier for a user or operator to understand the type of error encountered by the piece of equipment.

The list of error categories may contain error categories indicating different severity levels of respective error states. The list of error categories may include one or more of a first error category, indicating a high severity error; a second error category, indicating a medium severity error; and a third error category, indicating a low severity error. High severity errors may also be referred to as “red errors” and may include a failure of a piece of medical equipment requiring attention of an operator, a failure of a reprocessing procedure resulting in one or more endoscopes not being safe for use after the process, or the like. Medium severity errors may also be referred to as “yellow errors” and may include a status of a piece of medical equipment or a process parameter or the result of a procedure being outside an optimal range, but inside acceptable regulatory limits. Low severity errors may include advance notifications of device components approaching the end of their scheduled service time, process chemicals approaching the end of their shelf-life, or other upcoming maintenance activity.

The list of error categories may further contain error categories indicating whether or not the error state was caused by human error.

The error information data may contain data prompting an operator to perform an error handling procedure, the error handling routine being dependent from the one or more error categories assigned to the error state. The error information may further contain error resolution information specific to the piece of medical equipment affected by the error state.

The method may further comprise repeatedly receiving status information data from the one or more pieces of medical equipment, and terminating the outputting of the error information data when the status information indicates that the error state has been resolved.

The method may further comprise providing statistical information including one or more of:
a number or frequency of certain error states or errors of a certain error category within a predetermined or selectable time period;
an average or cumulated downtime caused by certain error states or errors of a certain error category and
a ranked list of most frequent error states or error categories.

The present disclosure further provides a method of handling medical equipment status information, the method comprising repeatedly receiving status information data from one or more pieces of medical equipment, storing the received status information data in a database; determining whether the status information data indicate an error state of a first one of the one or more pieces of medical equipment; and after determining that the status information data indicates an error state of the first one of the one or more pieces of medical equipment: analysing the stored status information data for first data patterns predicting the error state; searching stored status information data, received from at least a second one of the one or more pieces of medical equipment other than the first one, for second data patterns similar to the first data patterns; determining, based on a similarity between the second data patterns and the first data patterns, a probability that the second one of the one or more pieces of medical equipment will encounter an error state within a given time period; and when the probability exceeds a threshold, outputting predicted error information data comprising identification data identifying the second piece of medical equipment, status information data received from the second piece of medical equipment, and predicted error identification data identifying the predicted error state. By outputting predicted error information data, it is possible for a user to initiate countermeasures capable of avoiding the predicted error to occur.

The method may further comprise assigning, to each predicted error state, at least one predicted error category of a predetermined list of predicted error categories; and outputting the assigned predicted error category as part of the predicted error identification data. The list of predicted error categories may comprise predicted error categories indicating different severity levels of respective predicted error states.

The list of predicted error categories may comprise at least one predicted error category indicating that the predicted error state is unknown. Here, the term “unknown” is particularly meant to cover any error states which have not been fully analysed by a manufacturer of the affected piece of medical equipment.

At least one of the analysing, the searching, and the determining may employ one or more pattern recognition algorithms. At least one of the analysing, the searching, and the determining may be employed using an artificial neuronal network or a support vector machine.

The present disclosure further provides a system, comprising at least one endoscope reprocessing machine, a database, and a control unit (such as a controller, processor, CPU, circuit or computer), wherein the control unit is configured to perform a method as provided above.

### BRIEF DESCRIPTION OF THE DRAWINGS

In the following, some exemplary embodiments of the present disclosure are explained at using drawings. In the drawings:

FIG. 1 illustrates an endoscope,

FIG. 2 illustrates an endoscope reprocessing machine,

FIG. 3 illustrates a system for handling status data, and

FIG. 4 illustrates a schematic diagram of a computer-based error categorization system (ECS).

### DETAILED DESCRIPTION

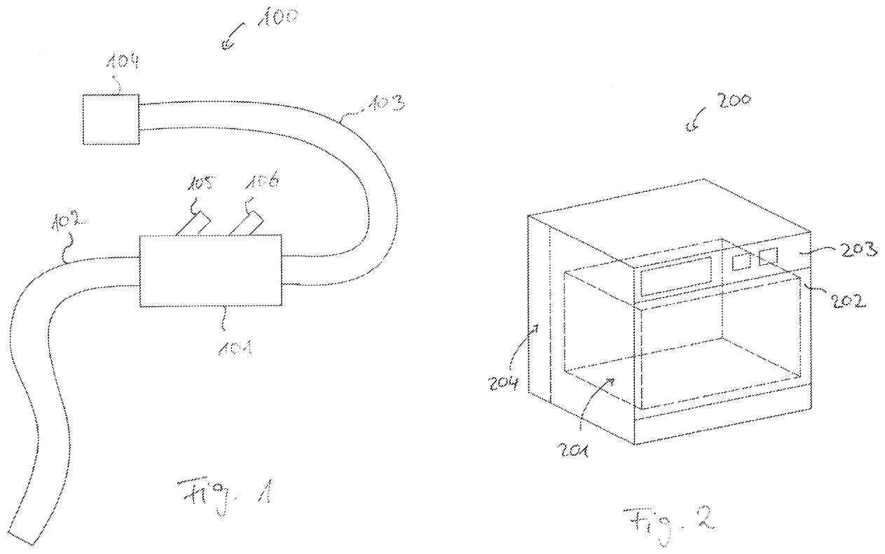

FIG. 1 shows an endoscope 100. The endoscope 100 comprises a main body 101, a flexible shaft 102, a supply cable 103, and a connector plug 104. On the main body 101, two treatment channel ports 105, 106 are provided. The treatment channel ports 105, 106 provide access to channels (not shown) in the flexible shaft 102, which end in a distal section of the flexible shaft 102. The channels allow insertion of surgical instruments through the flexible shaft 102 towards a surgical site, and the injection or aspiration of fluids.

Through the supply cable 103 and the connector plug 104, the endoscope 100 can be connected to a camera controller (not shown), which controls a video camera (not shown) in the distal end of the flexible shaft 102. The camera controller may also comprise a light source for coupling illumination light into the endoscope 100, which is emitted at the distal end of the shaft 102 for illuminating an area under examination or treatment.

Similar endoscopes are e.g., known from WO021/199180A1.

After each use, endoscopes like endoscope 100 have to be carefully reprocessed, before they can be used with another patient. Reprocessing involves mechanical and chemical cleaning of the outer surface of the endoscope 100 as well as any internal treatment channel. The chemical cleaning of endoscopes is performed in sophisticated endoscope reprocessing machines.

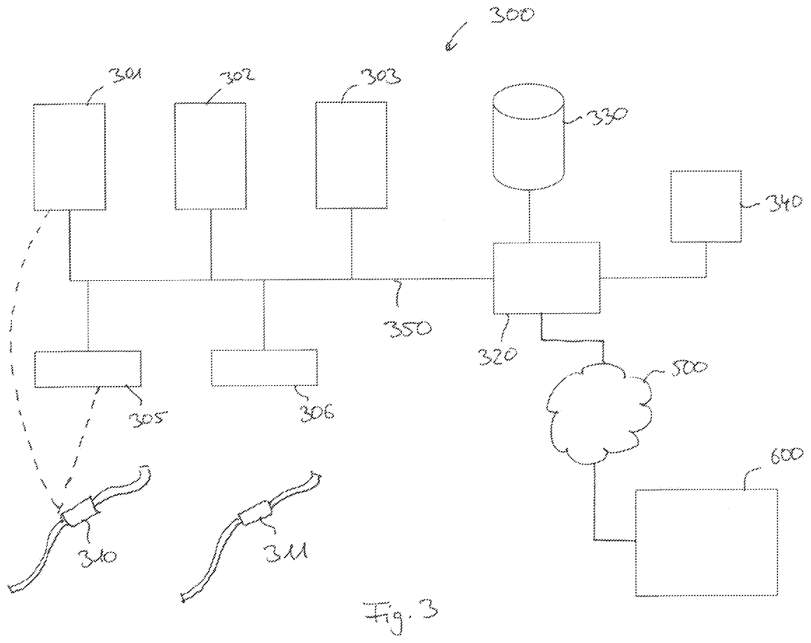

FIG. 2 shows one example of an endoscope reprocessing machine 200. The endoscope reprocessing machine 200 comprises an internal reprocessing chamber 201, which is accessible through a front door 201 for loading and unloading of endoscopes (not shown in FIG. 2). User controls 203 are provided at a front side of the endoscope reprocessing machine 200. The user controls 203 may include a display and one or more buttons for operating the endoscope reprocessing machines 200. At a rear side of the endoscope reprocessing machine 200, a storage compartment 204 is provided for storage of tanks of required chemicals like detergents and disinfectants. Connections for fresh and wastewater, electric supply and data communication are not shown.

Similar endoscope reprocessing machines are available from the applicant under the name “ETD”. The endoscope reprocessing machine 200 is just one possible example of endoscope reprocessing machines. Other available endoscope reprocessing machines include top-loading machines and machines having a clean-side/dirty-side design, wherein used endoscopes are loaded into the machine through a first door on a first (dirty) side, and are unloaded after processing through a second door on a second (clean) side. In the context of the present disclosure, the term “reprocessing machine” may also include drying cabinets used for drying the channels of endoscopes after reprocessing. Such drying cabinets are available from the applicant under the name “EDC Plus”.

The endoscope reprocessing machine 200 regularly provides status data concerning the machine itself and status data concerning endoscopes under reprocessing through a data connection. The status data is received by a control system.

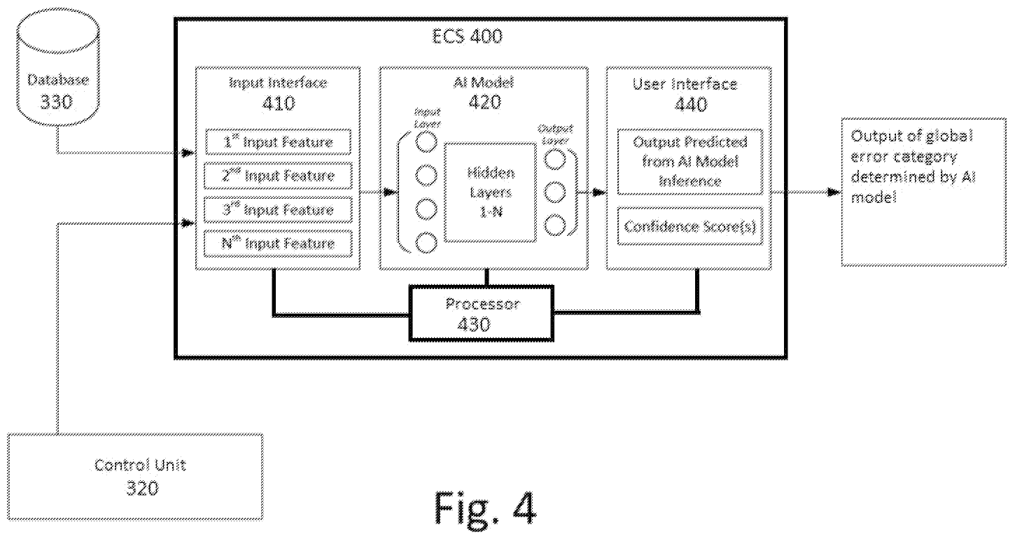

FIG. 3 shows a system 300 for handling status data for several pieces of medical equipment. The medical equipment includes a number of endoscope reprocessing machines 301, 302, 303, camera controllers 305, 306, and endoscopes 310, 311.

The system 300 further comprises a control unit 320 (such as a controller, processor, CPU, circuit or computer), a database 330, and a user interface 340 (such as a touchscreen or a display). The control unit 320 is connected to the endoscope reprocessing machines 301, 302, 303 and to the camera controllers 305, 306 through a wired or wireless network 350, for example a hospital network.

While not depicted in detail, the control unit 320 may employ standard computer hardware like a processor, memory, and bus systems for connecting the processor with the memory and other components. Such standard computer hardware is known to a person skilled in the art. In the memory, which may include one or more of Random-Acess-Memory (RAM), Read-Only-Memory (ROM), Flash-Memory, and Hard-Disk-Drives (HDD), computer readable instruction may be stored which cause the processor to execute any of the methods provided on this specification.

The database 330 may be an internal database hosted within the control unit 320, or an external database hosted on a database server separate from the control unit 320. When the database 330 is an external database, the database server may be a physical database server or a virtual database server. The database server may be provided on-premise, e.g., in a data center of a hospital, or off-premise, e.g., in a data center provided by a cloud computing service provider.

The endoscope reprocessing machines 301, 302, 303 and the camera controllers 305, 306 repeatedly transmit status information data through the network 350. The status information data is received by the control unit 320.

Status information data provided by the endoscope reprocessing machines 301, 302, 303 includes status information data relating to the respective endoscope reprocessing machine itself as well as status information data relating to individual endoscopes currently under reprocessing in the respective endoscope reprocessing machine.

A non-exhaustive list of status information data related to a reprocessing machine may include:
Time required to draw a specified amount of fresh water,
Time required to heat a reprocessing slurry to a predetermined temperature,
Dosage volume of detergent,
Dosage volume of disinfectant,

A non-exhaustive list of status information data related to an endoscope may include:
Endoscope identification data,
Endoscope type,
Maximum pressure drop during channel patency test,
Maximum leakage rate during leakage test.

Status information data provided by the camera controllers 305, 306 includes status information data related to endoscopes currently connected to the respective camera controller. For the present disclosure, the most relevant status information data includes data showing that a respective endoscope has been used together with a camera controller 305, 306, and is therefore due for reprocessing before being used with another patient. Status information data provided by the camera controllers 305, 306 may also include status information data related directly to the respective camera controller.

The control unit 320 stores status the received information data in the database 330, so that the status information data is available for later retrieval.

In case of errors during a reprocessing procedure, the respective endoscope reprocessing machine provides additional status information data containing an error code identifying the error. Errors may relate to a specific endoscope, or to a specific endoscope reprocessing machine.

A non-exhaustive list of endoscope-related errors may include:
Incomplete connection of adapter,
Failed leakage test,
Failed patency test.

A non-exhaustive list of machine-related errors may include:
Failure to reach target temperature,
Failure to draw predetermined volume of water,
Drainage failure,
Chemicals container not connected,
Insufficient chemicals reserve,
Basket coupling failure,
Door failure,
Failed identification of endoscope.

When the control unit 320 receives status information data indicating an error state, error information data in a form of an error message is provided to a user or operator of the system 300 through the user interface 340. The error information data includes identification data for identifying the piece of medical equipment that encountered the error state, like a machine identifying code or an endoscope identifying code. The error information data further includes status information data from the respective piece of medical equipment.

As the error code is specific for different types of endoscope reprocessing machines, it would be difficult of the user or operator to assess the importance of the error without referring e.g., to individual machine documentation. The control unit 320 therefore assigns at least one global error category from a list of global error categories to each error state, and includes the assigned global error categories into the error information data.

The global error categories are independent from a specific endoscope reprocessing machine, and may indicate the importance of a respective error. A non-exhaustive list of global error categories indicating importance of the error may include:
Very high importance (reprocessing procedure interrupted, manual intervention required),
High importance (reprocessing procedure completed, but invalid for one or more endoscopes),
Medium importance (reprocessing procedure completed, but operation parameters close to regulatory limits),
Low importance (reprocessing procedure successful, but manual intervention required before next procedure or in near future).

Examples of errors in the very high importance error category may include: door failure (e.g., incomplete closure of an automatic door), basket coupling failure, incomplete connection of adapter, failed leakage test, failure to reach target temperature, failure to draw predetermined volume of water, drainage failure, chemicals container not connected, and/or insufficient chemicals reserve. Examples of errors in the high importance error category may include: failed patency test and/or failed identification of endoscope. The very high importance error category and the high importance error category may be integrated into a single error category like a high importance error category. Errors of the high importance error category may be referred to as “red errors”.

Examples of errors in the medium importance error category may include: low pressure or temperature of fresh water intake resulting in increased dosing or heating time, and/or slow pressure build-up in the patency test due to foaming. Errors of the medium importance error category may be referred to as “yellow errors”.

Examples of errors in the low importance error category may include: advance information of a UV sterilization unit approaching the end of its service life, the contents of a chemicals container approaching the end of its shelf life, and/or upcoming scheduled maintenance activity like taking bio-samples from within a reprocessing machine. Errors of the low importance error category may also be referred to as “pre-notifications”.

The list of global error categories may further comprise error categories indicating whether an error is the result of a human error. Respective error categories may include:
Machine error, or
Human error.

For assigning the one or more global error categories to a specific error state, the control unit 320 may access information from the database 330. Various approaches may be applied for assigning global error categories to specific error states.

In a deterministic approach, a global error category can manually be assigned to each specific error code. A list of error codes and assigned error categories can than be stored in the database 330 in form of a look up table (LUT). After receiving an error code, the control unit 320 can access the LUT in the database 330 and read the corresponding error category from the LUT. Such approach may be most suitable for assigning global error categories indicating the severance of a specific error state.

In a non-deterministic approach using machine learning technologies, a global error category may be assigned to an error state through machine learning technologies. Such approach may for example be suitable for assigning global error categories indicating whether an error state results from human error. This is explained in more detail below with respect to FIG. 4.

FIG. 4 shows a schematic diagram of an exemplary computer-based error categorization system (ECS) 400 that is configured to assign a global error category to a specific error state based on status information data provided by a piece of medical equipment. In various embodiments, the ECS 400 includes an input interface 410 (such as a touchscreen, keyboard or mouse) through which status information data which are specific to an error state are provided as 1^{st} to N^{th} input features to an artificial intelligence (AI) model 420, a processor 430 which performs an inference operation in which the status information data are applied to the AI model 420 to generate the global error category, and a user interface (UI) 440 (such as a touchscreen or a display) through which the global error category is communicated to a user, e.g., an operator of the system 300. The UI 440 may be integral with the user interface 340 of the system 300. In some embodiments, the ECS 400 may be an integral part of the control unit 320.

In some embodiments, the input interface 410 may be a direct data link between the ECS 400 and the control unit 320. For example, the input interface 410 may status information data directly to the ECS 400 when an error state is encountered by any connected piece of medical equipment. Additionally, or alternatively, the input interface 410 may be a classical user interface that facilitates interaction between a user and the ECS 400. For example, the input interface 410 may facilitate a user interface through which the user may manually enter a global error category to be assigned to an error state during a learning phase of the ECS 400. Additionally, or alternatively, the input interface 410 may provide the ECS 400 with access to the database 330 to access historic status information of the same or a different piece of medical equipment stored in the database 330. In any of these cases, the input interface 410 may be configured to collect actual and historic status information data in association with a specific piece of medical equipment on or before a time at which the ECS 400 is used to assign a global error category to a specific error state.

Based on the status information data, the processor 430 performs an inference operation using the AI model 420 to generate a global error category to be assigned to the specific error state. For example, input interface 410 may deliver the status information data into an input layer of the AI model 420 which propagates these input features through the AI model to an output layer. The AI model 420 can provide a computer system the ability to perform tasks, without explicitly being programmed, by making inferences based on patterns found in the analysis of data. An AI model explores the study and construction of algorithms (e.g., machine-learning algorithms) that may learn from existing data and make predictions about new data. Such algorithms operate by building an AI model from example training data in order to make data-driven predictions or decisions expressed as outputs or assessments.

There are two common modes for machine learning (ML): supervised ML and unsupervised ML. Supervised ML uses prior knowledge (e.g., examples that correlate inputs to outputs or outcomes) to learn the relationships between the inputs and the outputs. The goal of supervised ML is to learn a function that, given some training data, best approximates the relationship between the training inputs and outputs so that the ML model can implement the same relationships when given inputs to generate the corresponding outputs. Unsupervised ML is the training of an ML algorithm using information that is neither classified nor labelled, and allowing the algorithm to act on that information without guidance. Unsupervised ML is useful in exploratory analysis because it can automatically identify structure in data.

Common tasks for supervised ML are classification problems and regression problems. Classification problems, also referred to as categorization problems, aim at classifying items into one of several category values (for example, is this object an apple or an orange?). Regression algorithms aim at quantifying some items (for example, by providing a score to the value of some input). Some examples of commonly used supervised-ML algorithms are Logistic Regression (LR), Naive-Bayes, Random Forest (RF), neural networks (NN), deep neural networks (DNN), matrix factorization, and Support Vector Machines (SVM).

Some common tasks for unsupervised ML include clustering, representation learning, and density estimation. Some examples of commonly used unsupervised-ML algorithms are K-means clustering, principal component analysis, and autoencoders.

Another type of ML is federated learning (also known as collaborative learning) that trains an algorithm across multiple decentralized devices holding local data, without exchanging the data. This approach stands in contrast to traditional centralized machine-learning techniques where all the local datasets are uploaded to one server, as well as to more classical decentralized approaches which often assume that local data samples are identically distributed. Federated learning enables multiple actors to build a common, robust machine learning model without sharing data, thus allowing to address critical issues such as data privacy, data security, data access rights and access to heterogeneous data.

In some examples, the AI model 420 may be trained continuously or periodically prior to performance of the inference operation by the processor 430. Then, during the inference operation, the status information data provided to the AI model 420 may be propagated from an input layer, through one or more hidden layers, and ultimately to an output layer that corresponds to the global error category to be assigned to a specific error state. For example, if an endoscope reprocessing machine 301, 302, 303 reports an error because a connecting force necessary to fully connect an endoscope basket to an irrigation system connector exceeds a threshold, the AI model 420 may use historic data like the last time an adapter plate of the basket has been changed, connection forces measured during previous coupling operations, and the like, for determining whether the error state is the result of a human error (e.g., incorrect placement of the adapter plate) or a machine error (e.g., degradation of seals, or the like), and provide a respective global error category as an output. Together with the determined global error category, the AI model 420 may provide a confidence level of the determination, depending on the training quality of the AI model 420. If the confidence level provided does not meet a predetermined threshold, a user or operator may be prompted to manually analyse the status data to determine whether the error state results from human or machine error, and to provide the result of the manual determination to the AI model 420 though the input interface 410. Such input may then be used to further train the AI model 420 so that future inference operations may return better confidence levels.

During and/or subsequent to the inference operation, the global error category may be communicated to the control unit 320 for inclusion into the error information data to be provided to the user or operator.

The control unit 320 may use a combination of deterministic and non-deterministic approaches as described above for assigning the one or more global error categories to a specific error state.

Further to the global error category, the error information data may include data prompting an operator to perform an error handling procedure depending on the one or more error categories. Error handling procedures may include one or more of: recording predefined or editable log entries in log records of the piece of medical equipment that encountered the error state; marking all or some endoscopes involved in an affected reprocessing procedure as due for renewed reprocessing; and marking an endoscope reprocessing machine affected by the error state as due for maintenance before start of next reprocessing procedure.

The error information data may further comprise error resolution information specific to the piece of medical equipment that encountered the error state. For example, if an endoscope reprocessing machine 301, 302, 303 reports an error because a connecting force necessary to fully connect an endoscope basket to an irrigation system connector exceeds a threshold, the error information data provided by the control unit 320 may include a copy of or a link to a section of an operator's manual of the affected endoscope reprocessing machine that deals with checking or correcting the function of the basket coupling.

Independently from outputting the error information data through the user interface 340, the control unit 320 continues receiving status information data from the one or more pieces of medical equipment. If, for a piece of equipment that previously reported an error state, the actual status information data no longer indicate an error state, the outputting of the error information data is terminated, i.e., the error message is cleared from the user interface 340.

Further to outputting error information data related to individual error states, the control unit 320 may also compute statistical data related to error states. Such statistical data may include the number or the frequency of certain error states, or errors of a certain global error category. For example, a count of error states being categorized as “very high importance” within the last week, month, calendar quarter, or other selectable period, may be provided as textual or graphical information through the user interface 340, or may be stored in a global error log in the database 330. Similarly, a cumulated or average downtime related to a certain error state or a certain global error category may be computed, displayed, and stored. For example, a cumulated downtime of endoscope reprocessing machines due to human error may be computed and displayed. Such information may then be used by management to determine the necessity of certain operator trainings so that the downtime caused by human error can be reduced.

It is further possible to compute, display and store a ranking of certain error states or global error categories according to their frequency and/or the downtime caused by respective error states or global error categories. Such information may be used by management for focussing improvement measures.

The control unit 320 may further be configured to communicate status information data from pieces of medical equipment in the system 300 through a global communication network 500, e.g., the internet, to an external data center 600. Between the control unit 320 and the communication network 500, a firewall (not shown) or other protection against malicious communication content may be provided.

In the external data center 600, a computer-based error analysis and prediction system EAPS may be implemented. The basic structure and function of the EAPS may be very similar to the structure and function of the ECS 400 described above, so that a detailed description is omitted here for brevity. The EAPS can employ ML for identifying patterns in status information data preceding a transition of a piece of medical equipment from a regular (non-error) state into an error state.

The status information data and the error code corresponding to the error state are provided to the EAPS as training data, wherein the status information data is provided as input, and the error code of the encountered error state is provided as target output. By repeated training, the EAPS will “learn”, e.g., by application of pattern recognition algorithms, to identify data patterns in the status information data that predict the transition into the respective error state. The described learning method formally qualifies as “unsupervised learning”, as the training data does not comprise annotations provided by human experts. However, the error codes provided through instances outside of the EAPS have a similar function as human expert annotations, so that the training method is more similar to “supervised learning”, and respective algorithms may be applied. The EAPS may employ an artificial neuronal network or a support vector machine.

After sufficient training, the EAPS can then be used to recognize similar data patterns in the status information data received from other pieces of medical equipment, which can be installed at different facilities. Based on the recognition, the EAPS can predict that a certain error state will be encountered by a certain piece of medical equipment at some time in the future, and with a certain probability. Such prediction result can then be provided back to the control system 320, which can compile and output predicted error information data through the user interface 340.

The predicted error information data may comprise identification data identifying the piece of medical equipment for which the error is predicted, status information data received from the piece of medical equipment for which the error is predicted, and predicted error identification data identifying the predicted error state. The predicted error identification data may comprise an error code of the predicted error state.

Similar as described above with respect to error messages, the control unit 320 may assign one or more predicted error categories from a list of predicted error categories to the predicted error states, to make the predicted error information data easier understandable for a user or operator of the system 300. The list of predicted error categories may include predicted error categories indicating different severity levels of the predicted error states. The list of predicted error categories may further include predicted error categories indicating whether or not a predicted error state is unknown. In the context of the present disclosure, an “unknown predicted error state” is understood to be a predicted error state the nature of which isn't yet fully analysed.

The EAPS may be trained or “hard-wired” to recognize error-predicting data patterns that are fully understood. For example, if the time period required to heat a fluid reservoir to a target temperature in a certain endoscope reprocessing machine slowly increases, the EAPS can predict that, in future, the respective endoscope reprocessing machine will encounter an error state due to the fluid reservoir not reaching the target temperature within an allowed time period.

For known predicted error states, the predicted error state information data may further comprise information regarding possible error prevention measures.

However, if a new generation of medical equipment is released into the market, not all failure mechanisms may be fully analysed. Here, the EAPS may be used to detect data patterns indicating new failure mechanisms early, which can then be used to further investigate such failure mechanisms. At the same time, the EAPS can learn to predict “unknown” error states, so that a user of the system 300 can take appropriate countermeasures to accommodate for the expected failure of the equipment.

For example, if one or more endoscopes of a certain type fail a leakage test prematurely, i.e., before the endoscope has reached the expected end of its lifetime, the EAPS may analyse the status information data from those endoscopes in the time before the failure, and detect data patterns predicting the failure.

As a pure fictive example, the EAPS may detect that the respective endoscopes fail after being reprocessed several times using a reprocessing chemical from a certain production lot, but do not fail after being reprocessed the same number of times with the same reprocessing chemical from a different production lot. The EAPS can then analyse status information from the same or other facilities and prompt the respective control units to present predicted error information data to a user or operator, when endoscopes of the affected type undergo reprocessing with the reprocessing chemical from the affected production lot.

As a further fictive example, the EAPS may detect that a certain type of endoscopes often encounters an error due to connection hoses slipping off the endoscope connectors if the endoscope gets reprocessed in a particular type of endoscope reprocessing machines, or in a particular reprocessing position within an endoscope reprocessing machine, like position 1, position 2, or the like. The EAPS may then analyse status information from the same or other facilities and prompt respective control units to present predicted error information data to a user or operator, when an endoscope of the affected type is scheduled to be reprocessed in the affected reprocessing machine type and/or on the affected reprocessing position. In this particular situation, the predicted error information data may include a reminder to apply specific caution when connecting the endoscope to the connection hoses.

While there has been shown and described what is considered to be preferred embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

What is claimed is:

1. A method of handling medical equipment status information, the method comprising:
receiving status information data from two or more pieces of medical equipment, wherein the status information data is received in a data format specific for each of the two or more pieces of medical equipment;
determining that the status information data indicate an error state of the two or more pieces of medical equipment;
assigning to each error state one or more global error categories of a predetermined list of global error categories, the global error categories not being specific to a certain piece of medical equipment among the two or more pieces of medical equipment; and
outputting error information data comprising:
identification data identifying a specific piece of medical equipment among the two or more pieces of medical equipment,
status information data received from the specific piece of medical equipment, and
the one or more global error categories assigned to the detected error status.

2. The method of claim 1, wherein the list of error categories contains error categories indicating different severity levels of respective error states.

3. The method of claim 2, wherein the list of error categories includes one or more of:
a first error category, indicating a high severity error, and
a second error category, indicating a medium severity error.

4. The method of claim 3, wherein the list of error categories further includes a third error category, indicating a low severity error.

5. The method of claim 1, wherein the list of error categories contains error categories indicating whether or not the error state was caused by human error.

6. The method of claim 1, wherein the error information data contains data prompting an operator to perform an error handling procedure, the error handling procedure being dependent from the one or more error categories assigned to the error state.

7. The method of claim 6, wherein the error information data further contains error resolution information specific to the piece of medical equipment affected by the error state.

8. The method of claim 1, further comprising:
repeatedly receiving status information data from the two or more pieces of medical equipment, and
terminating the outputting of the error information data when the status information indicates that the error state has been resolved.

9. The method according to claim 1, further comprising providing statistical information including one or more of:
a number or frequency of certain error states or errors of a certain error category within a predetermined or selectable time period,
an average or cumulated downtime caused by certain error states or errors of a certain error category, and
a ranked list of most frequent error states or error categories.

10. A method of handling medical equipment status information, the method comprising:
repeatedly receiving status information data from two or more pieces of medical equipment,
storing the received status information data in a database,
determining whether the status information data indicate an error state of a first one of the two or more pieces of medical equipment; and
after determining that the status information data indicates an error state of the first one of the two or more pieces of medical equipment:
analysing the stored status information data for first data patterns predicting the error state;
searching stored status information data, received from at least a second one of the two or more pieces of medical equipment other than the first one of the two or more pieces of medical equipment, for second data patterns similar to the first data patterns;
determining, based on a similarity between the second data patterns and the first data patterns, a probability that the second one of the two or more pieces of medical equipment will encounter an error state within a given time period; and
when the probability exceeds a threshold, outputting predicted error information data comprising:
identification data identifying the second one of the two or more pieces of medical equipment,
status information data received from the second one of the two or more pieces of medical equipment, and
predicted error identification data identifying the predicted error state.

11. The method of claim 10, further comprising:
assigning, to each predicted error state, at least one predicted error category of a predetermined list of predicted error categories; and
outputting the assigned predicted error category as part of the predicted error identification data.

12. The method of claim 11, wherein the list of predicted error categories comprises predicted error categories indicating different severity levels of respective predicted error states.

13. The method of claim 11, wherein the list of predicted error categories comprises at least one predicted error category indicating that the predicted error state is unknown.

14. The method of claim 10, wherein at least one of the analysing step, the searching step, and the determining step employ one or more pattern recognition algorithms.

15. The method of claim 10, wherein at least one of the analysing step, the searching step, and the determining step are employed using one of an artificial neuronal network and a support vector machine.

16. A system, comprising
at least one endoscope reprocessing machine,
a database, and
a controller configured to:
receive status information data from two or more pieces of medical equipment, wherein the status information data is received in a data format specific for each of the two or more pieces of medical equipment;
determine that the status information data indicate an error state of the two or more pieces of medical equipment;
assign to each error state one or more global error categories of a predetermined list of global error categories, the global error categories not being specific to a certain piece of medical equipment among the two or more pieces of medical equipment; and
output error information data comprising:
identification data identifying a specific piece of medical equipment among the two or more pieces of medical equipment,
status information data received from the specific piece of medical equipment, and
the one or more global error categories assigned to the detected error status.

17. A system, comprising
at least one endoscope reprocessing machine,
a database, and
a controller configured to:
repeatedly receive status information data from two or more pieces of medical equipment,
store the received status information data in a database,
determine whether the status information data indicate an error state of a first one of the two or more pieces of medical equipment; and
after determining that the status information data indicates an error state of the first one of the two or more pieces of medical equipment:
analyse the stored status information data for first data patterns predicting the error state;
search stored status information data, received from at least a second one of the two or more pieces of medical equipment other than the first one of the two or more pieces of medical equipment, for second data patterns similar to the first data patterns;
determine, based on a similarity between the second data patterns and the first data patterns, a probability that the second one of the two or more pieces of medical equipment will encounter an error state within a given time period; and
when the probability exceeds a threshold, output predicted error information data comprising:
identification data identifying the second one of the two or more pieces of medical equipment,
status information data received from the second one of the two or more pieces of medical equipment, and
predicted error identification data identifying the predicted error state.
